(19) United States
(12) Patent Application Publication
(10) Pub. No.: US 2023/0165240 A1
(43) Pub. Date: Jun. 1, 2023

(54) DEVICE FOR PERFUSION AND PRESERVATION OF TISSUE SPECIMENS EX VIVO

(71) Applicant: IMPERIAL COLLEGE INNOVATIONS LIMITED, London (GB)

(72) Inventors: Darryl OVERBY, London (GB); Joseph VAN BATENBURG-SHERWOOD, London (GB); Foivos CHATZIDIMITRIOU, London (GB)

(21) Appl. No.: 17/906,602

(22) PCT Filed: Mar. 16, 2021
(86) PCT No.: PCT/EP2021/056739

§ 371 Date: Sep. 16, 2022

(30) Foreign Application Priority Data
Mar. 16, 2020 (GR) 20200100136
Mar. 23, 2020 (GB) 2004177.8

(51) Int. Cl. A01N 1/02

(52) U.S. Cl. CPC A01N 1/0247

(57) ABSTRACT

The present application relates to methods for perfusing a biological sample in a device comprising a channel comprising a constriction wherein the method comprises flowing a liquid through the channel, sealing the channel with the biological sample and maintaining a flow of liquid within the biological sample. The application also relates to a method for assaying a substance, a device for use in perfusing a biological sample and a method of manufacturing a device.

The present application relates to methods for perfusing a biological sample. The methods involve a device configured to maintain a flow of liquid within the biological sample.

In medical practice, clinicians collect tissue specimens from patients to perform tests that determine whether a tissue is physiologic or not. For example, in case of malignancy, histological analysis will inform patient treatment according to the stage and molecular profile of the tumour. To preserve cell identifying features, pathologists process tissue samples with fixatives, a process that stops proteolytic degradation, but also kills the cells. Although fixed specimens can be used for clinical examination they no longer actively respond to external stimuli, limiting their applicability for personalised therapy applications. Since primary sample use is the optimum approach for studying the characteristics of a patient's condition, the need for laboratory means to preserve ex vivo specimens remains unmet.

Similarly, to unravel the mechanisms shaping disease progression and the potential benefit of a given medical intervention, researchers widely use laboratory platforms that mimic physiological systems within the human body. Currently, numerous examples of such platforms are commercially available that comprise primary or cell line-derived cells, spheroids or microtissues together with several stimuli necessary for cell well-being such as shear stress, extracellular matrix proteins and growth factors. However, bottom-up construction of in vitro systems and lack of complex tissue-mimicking structures limit signalling pathways between cells and the ability to study patient-relevant phenotypes. Indeed, despite the increasing application of these models in research and drug development, such platforms are considered to be poor predictors of how the organism responds to a given agent.

Whilst developing a high-relevance in vitro model is challenging and expensive, tissue samples comprise practically all structural features of the in vivo environment. However, tissue lifetime ex vivo is extremely limited, mainly due to the difficulty in supplying nutrients and oxygen within it, and the absence of vascular perfusion. Tissue explants are commonly cultured while submerged in culture media, where mass transport is exclusively restricted to diffusion, through the tissue surface. Diffusion may be slow and due to consumption by cells delivery of nutrients is very limited beyond 100 μm. This major difference between ex vivo and in vivo situations results in reduced explant viability, insignificant response to external stimuli and technical difficulties in biological assay performance.

For these reasons, overcoming diffusion-limited mass transport limitations is an emerging need in several fields that would benefit from the wider use of tissue specimens in preclinical and clinical assays, leading to more robust laboratory models and in turn reduced animal use for research. Disclosed herein is a method that provides perfusion of tissue samples through the vasculature of a tissue sample, which involves a device comprising a constriction.

### SUMMARY OF THE INVENTION

Described herein is a method for perfusing a biological sample in a device comprising a channel comprising a constriction. The method provides quicker delivery and deeper penetration of oxygen and substances in a biological sample. This contributes towards longer preservation. In addition to increased sample viability ex vivo, this method can be used as a platform for more in vivo relevant studies of responses to a medical intervention. The method provides a more realistic model to study the effects of substances on tissues.

According to a first aspect, the invention provides a method for perfusing a biological sample in a device comprising a channel comprising a constriction wherein the method comprises flowing a liquid through the channel, sealing the channel with the biological sample and maintaining a flow of liquid within the biological sample.

According to a second aspect, the invention provides a method for assaying a substance comprising maintaining a flow of liquid within a biological sample by a method according to any preceding claim, wherein the liquid comprises the substance and detecting one or more effects of the substance on the biological sample.

According to a third aspect, the invention provides a device for use in perfusing a biological sample, wherein the device comprises a channel comprising a constriction wherein the constriction has a width of up to around 0.65 mm, a height around 0.75 mm, a Height to Width ratio of up to about 1.15 and/or a cross-sectional area of up to about 0.44 mm².

According to a fourth aspect, the invention provides method of manufacturing a device according to the third aspect, the method comprising bonding a flat surface of a first layer to a flat surface of a second layer, wherein the flat surface of the second layer comprises a groove, wherein the channel is formed by the groove and the flat surface of the first layer.

### REFERENCE IS MADE TO A NUMBER OF FIGURES AS FOLLOWS

FIG. 1. DESIGN & FABRICATION—3D printed moulds designed with CAD software were used to fabricate polydimethylsiloxane (PDMS) channels. The device comprises three layers: 1) PDMS reservoir 2) PDMS layer with channel architecture 3) Flat PDMS bottom. Layer are bonded one to the other through plasma treatment (45 seconds on each surface) and 3 hour treatment in an oven). Tubing is connected to the device and luer adapters with PDMS that is cured in an oven overnight (65° C.). (a-c): Constriction region where tissue specimen gets immobilised. Suitable shape and dimensions allow specimen immobilisation without severely limiting tissue permeability. (d-f): Device filled with liquid.

FIG. 2. DEVICE FOR PERFUSION OF NATIVE TISSUE SPECIMENS: (I) (A) multi-layer fabrication process, mould and polymerised PDMS layer for the channel (B, D respectively) and the reservoirs (C,E), (F) a 3 mm mouse liver specimen immobilised at the device constriction region, with inset showing the front view of a fully-assembled device filled with saline with food colouring. (II) Tissue specimen loading and immobilisation within the constriction under flow result in blood washout as confirmed by light microscopy (right: blood washout image scale: 10x, inset image scale: 40×).(iii) Visualisation of fluid flow using fluorescent tracers in a perfused and a peri-fused specimen. Specimen size on both cases was 3 mm. Arrows point to flow direction.

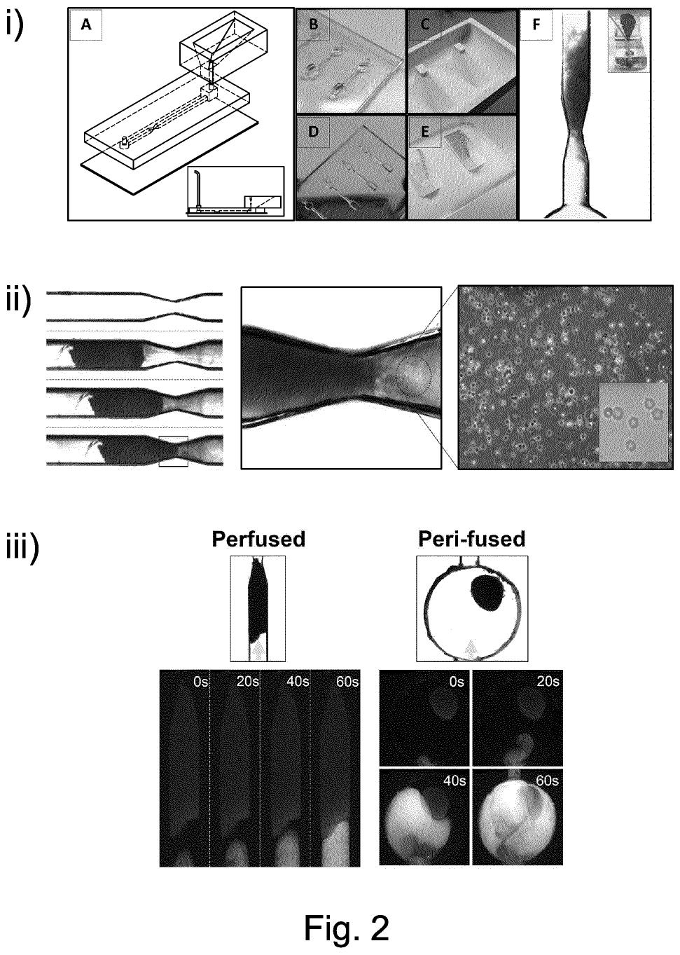

FIG. 3. DEVICE USE FOR EX VIVO TISSUE SPECIMEN PERFUSION—Tissue specimens are collected with a 3-mm sterile biopsy punch. Each tissue sample is loaded into the inlet reservoir and directed to the constriction site by flow using a syringe pump. This enables the sample to seal up the constriction region, restricting flow around it. Using a syringe pump a pressure gradient is then generated across the tissue sample, driving flow through it.

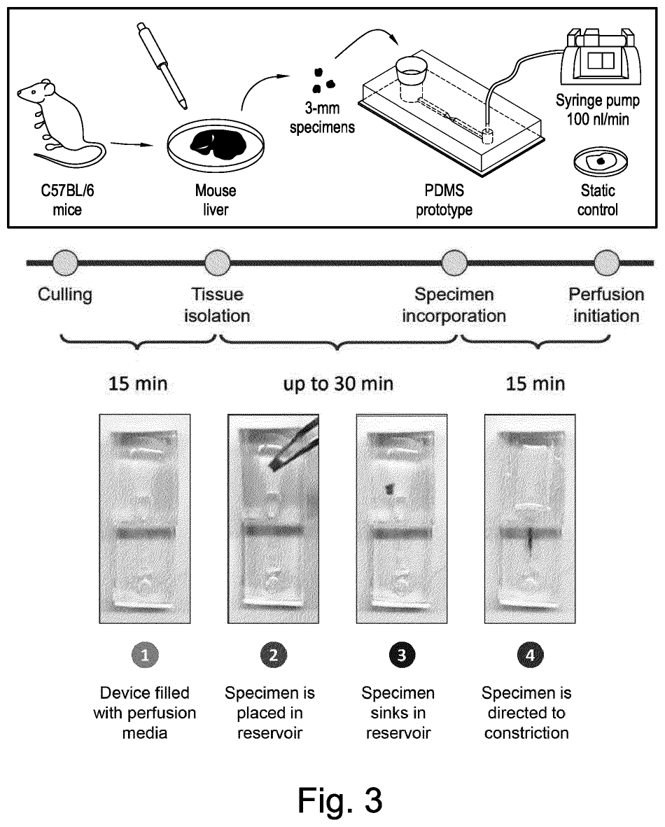

FIG. 4. INTRA-TISSUE FLOW DEMONSTRATION (1)—(Top) Confocal microscopy on 10 μm frozen sections of perfused mouse liver specimens. 0.2 μm red fluorescent tracers were located within the core of the perfused specimen. (Bottom) CD31 (green) and nuclei (blue) staining, and 0.2 μm polystyrene beads (red) on perfused and statically cultured specimen cryosections—Scale: 50 μm.

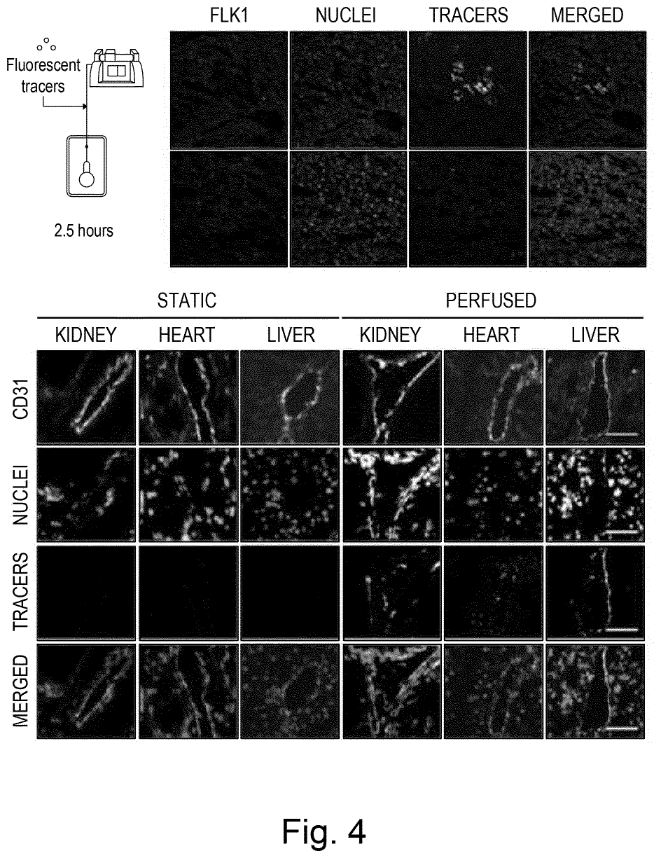

FIG. 5. INTRA-TISSUE FLOW DEMONSTRATION (2)—(A) Cell morphology in perfused, peri-fused, static and compression control cases after 2h of perfusion (scale bar: 8 μm), where F-actin is red, cell nuclei are grey and cytoplasm is pseudocoloured green (using cell autofluorescence), The arrow indicates flow direction for the perfused and peri-fused conditions, (B) Schematic of WGA use for each culture condition, (C) Graph showing fluorescence intensity for WGA in each specimen group (perfused versus static: p=0.035; perfused versus peri-fused: p=0.049; static versus perk fused: p=0.068). (D) Representative images for perfused, peri-fused and static samples, showing WGA in red and cell nuclei in grey (scale bar: 40 μm).

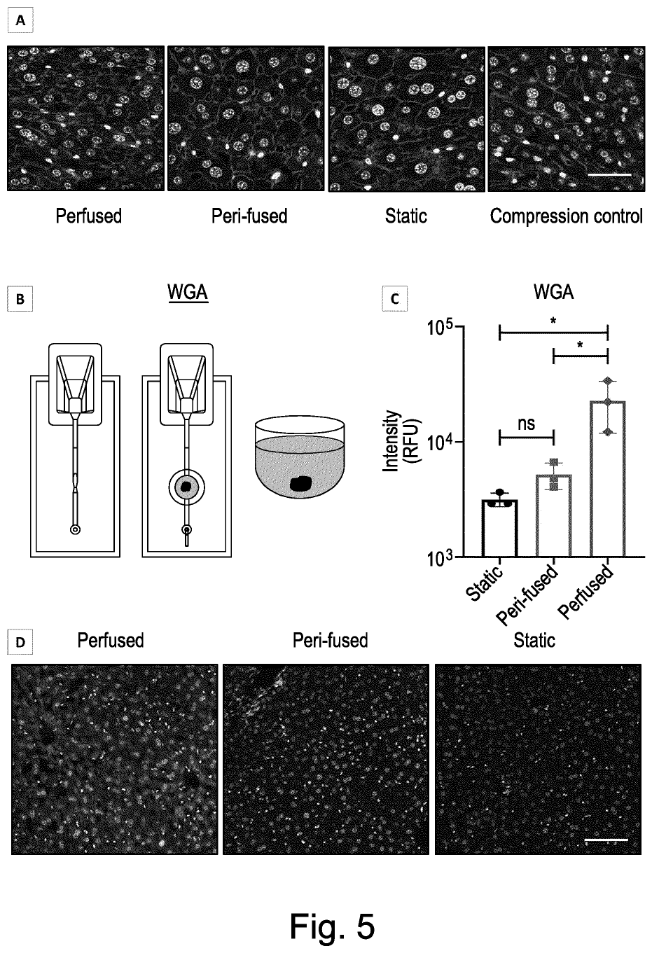

FIG. 6. INTRA-TISSUE FLOW DEMONSTRATION (3) (A) Experimental protocol for perfusion efficacy assessment using fluorescent particles, including a static culture stage before dividing specimens to different culturing conditions (B) Fuorescence intensity measurement in the lysate of perfused, peri-fused and static samples (perfused versus static: p=0.002; perfused versus peri-fused: p=0.0004; static versus peri-fused: p=0.06) (C) Fluorescent particle imaging on specimen cryosections fluorescent tracers are present within perfused but not peri-fused or static specimens. Nuclei are stained with Hoechst 33342, cytoplasm is psedocoloured using cell autofluorescence and polystyrene beads are fluorescent in the red channel, scale bar: 25 μm Inset: stained with Hoechst 33342, the vasculature is fluorescently tagged (Tie2-GFP) and polystyrene beads are red, inset scale bar: 25 μm. Throughout section C arrows point to fluorescent tracers (D) Live tracer imaging within a perfused specimen of a Tie2-GFP mouse (E) Imaging of fluorescent particles within a zebrafish skeletal muscle sample.

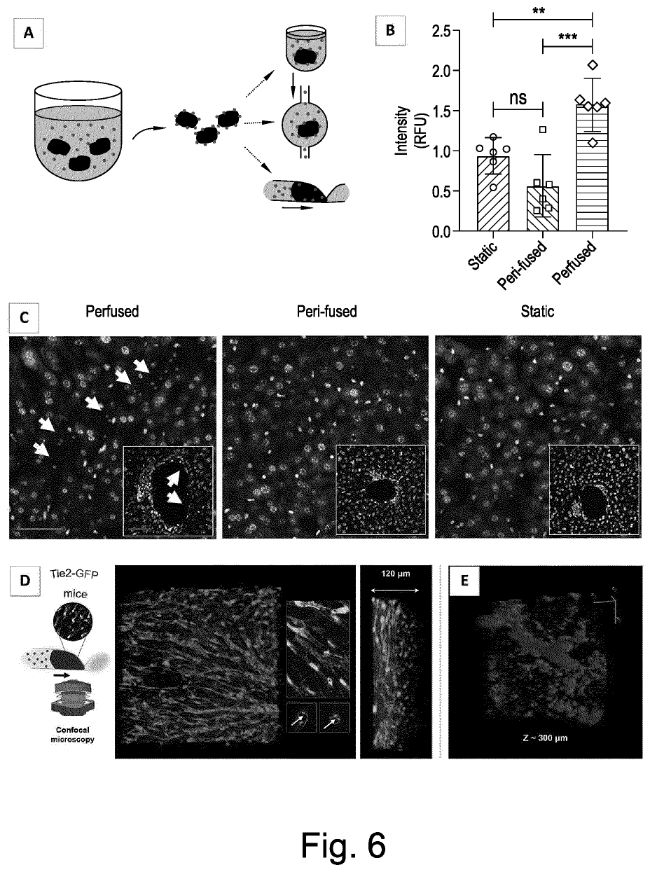

FIG. 7. INTRA-TISSUE FLOW DEMONSTRATION (4) (A) Specimen processing whilst being in the constriction for paraffin infiltration and embedment (B) COMSOL fluid flow simulation within an isotropic porous solid (C) Representative microCT sections from different specimen regions (Scale: 0.5 mm) (D) 3D reconstruction of microCT slices for the whole tissue part within the constriction (E) 150-μm thick (median) slice of the 3D view (F) Single-slice, transverse diagonal view through the 3D view.

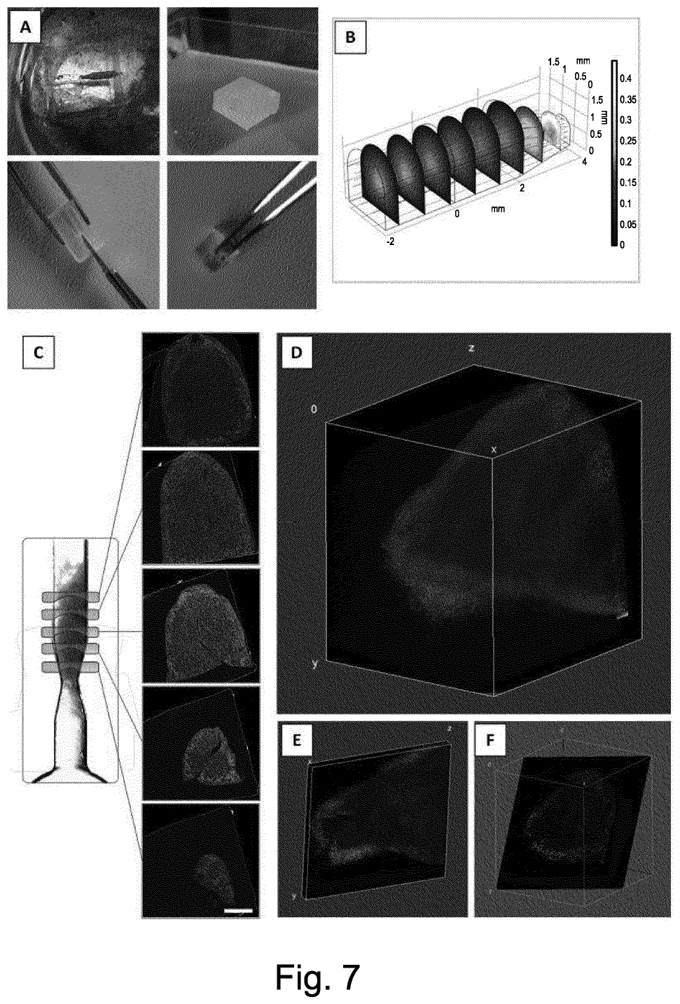

FIG. 8. INTRA-TISSUE FLOW DEMONSTRATION IN PERFUSED TISSUE SPECIMENS IN 3D (A): Confocal imaging on perfused zebrafish skeletal muscle specimen after perfusion with 0.2 μm red fluorescent tracers for 2.5 hours at 200 nl/min. Reconstructed slice thickness: 150 μm (B) Micro-computed tomography (pCT) on mouse liver specimen perfused with 20 nm gold nanoparticles for 2.5 hours.

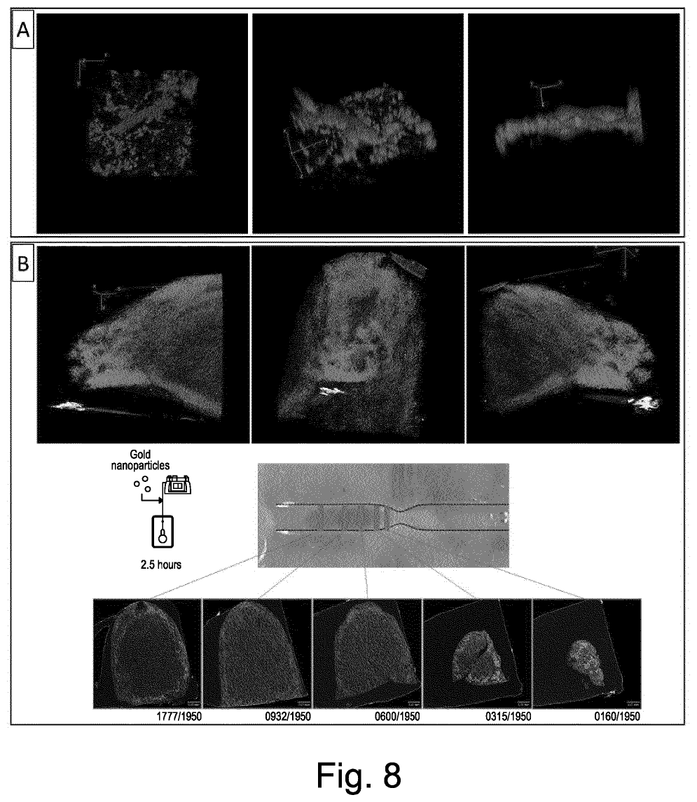

FIG. 9. PERFUSION EFFECT ON VIABILITY (1) Viability of murine liver explants after 48 h of perfusion, peri-fusion or static culture as measured by intracellular retention of total LDH per specimen (A) (perfused versus static: p<0.0001; perfused versus peri-fused: p=0.045; static versus peri-fused: p<0.0001), normalised LDH to protein (B) (perfused versus static: p<0.0001; perfused versus peri-fused: p<0.0001; static versus peri-fused: p=0.00012), total ATP per specimen (C) (perfused versus static: p<0.0001; perfused versus peri-fused: p<0.0001; static versus peri-fused: p=0.63) and normalised ATP to protein (D) (perfused versus static: p<0.0001; perfused versus perk fused: p<0.0001; static versus peri-fused: p=0.68).

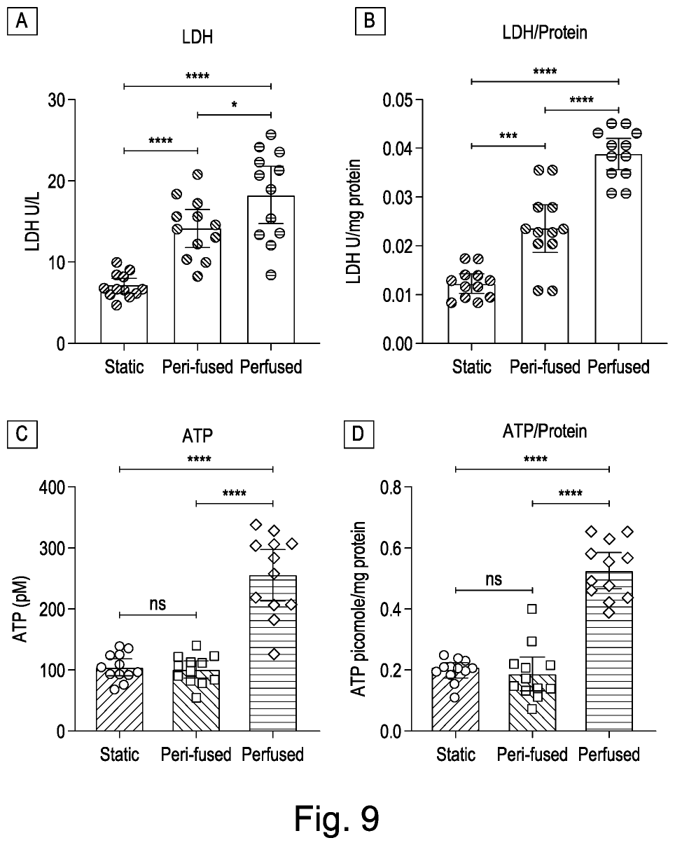

FIG. 10. PERFUSION EFFECT ON VIABILITY (2)—10 μm frozen mouse liver sections stained for cleaved caspase 3 (apoptotic marker) and Haematoxylin. The perfused sample presented less cleaved-caspase 3 positive cells that negative, static and perfisued controls after a 48 h tissue culture in a CO₂ incubator. (*) NC: Negative control—static tissue culture in saline only, PC: Positive control—Fresh tissue sample fixed immediately after isolation, Static: Static tissue culture in media, Peri-fused: Tissue specimen cultured in a static chamber where media was renewed at 100 nl/min, Perfused: Perfused tissue culture in the device with the same media as the static control.

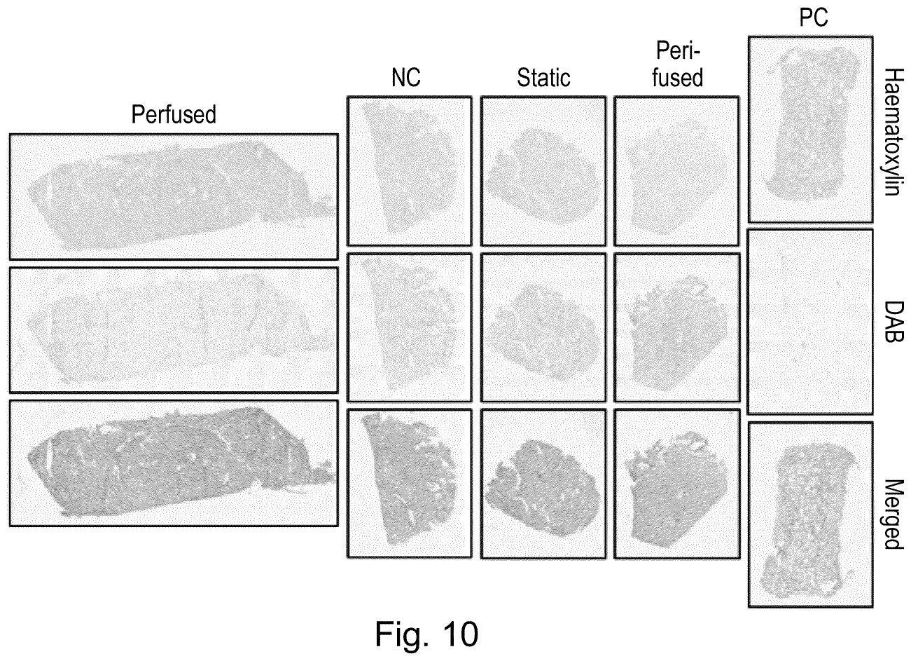

FIG. 11. PERFUSION EFFECT ON LIPID CONTENT & FLOW RATE OPTIMISATION—Oil Red O staining on 10 μm frozen sections of mouse liver specimens. Perfusion at 100 nl/min preserved cell lipid content after a 24 h tissue culture in a CO₂ incubator. 100 nl/min was superior to 200 nl/min for lipid content maintenance after 24 hours of perfusion. (*) Positive control: Fresh tissue sample fixed immediately after isolation, Perfused: Perfused tissue culture in the device.

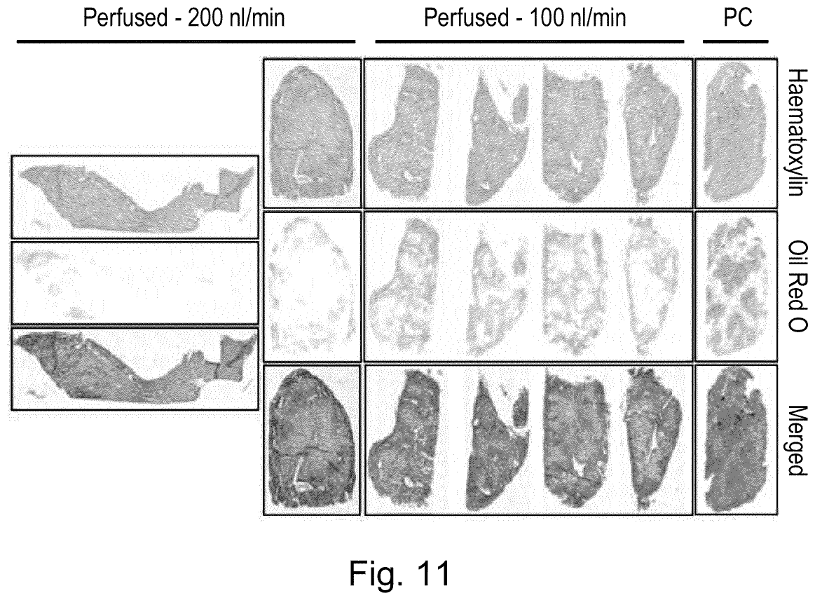

FIG. 12. TISSUE RESPONSE TO A METABOLIC POISON AFTER 2 H—3-mm mouse liver specimens were treated with a metabolic poison or control mixture for 2 h in perfused, peri-fused or static conditions in a CO₂ incubator. (A) ATP quantification in specimen lysates, showing that perfusion in this device resulted in higher metabolic inhibition in liver specimens (B) Oil Red O staining and Hematoxylin on 10 μm mouse sections demonstrate more profound differences for cell lipid content between perfused samples with the metabolic poison and perfused samples with control mixture (C) Periodic-Acid-Schiff's staining shows perfused specimen carbohydrate levels were affected by perfusion with a metabolic poison—Fresh tissue sample fixed immediately after isolation, Static: Static tissue culture in media, Peri-fused: Tissue specimen cultured in a static chamber where media was renewed at 100 nl/min, Perfused: Perfused tissue culture in the device.

FIG. 13. PRESERVATION OF MOUSE AND HUMAN OMENTAL TUMOURS—(A) 10 μm formaldehyde-fixed paraffin embedded mouse omental tissue sections stained for Hematoxylin & Eosin, Cleaved caspase 3 and WT1 (DAB used as chromogen). Lower cleaved caspase 3 signal suggest lower apoptosis and better maintenance for the perfused specimens. (B) 10 μm cryosections of human omental tumours stained for Cleaved caspase 3. Lower cleaved caspase 3 signal suggest lower apoptosis and better maintenance for the perfused specimens.

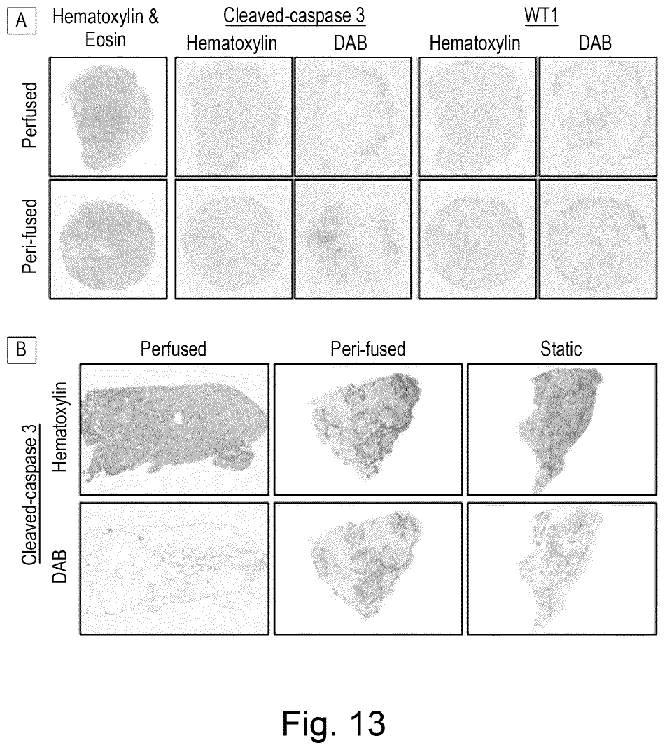

FIG. 14. HUMAN COLON SPECIMEN TREATMENT WITH SHORT CHAIN FATTY ACIDS—Example of an application of this device in the perfusion of human colon specimens with short chain fatty acids

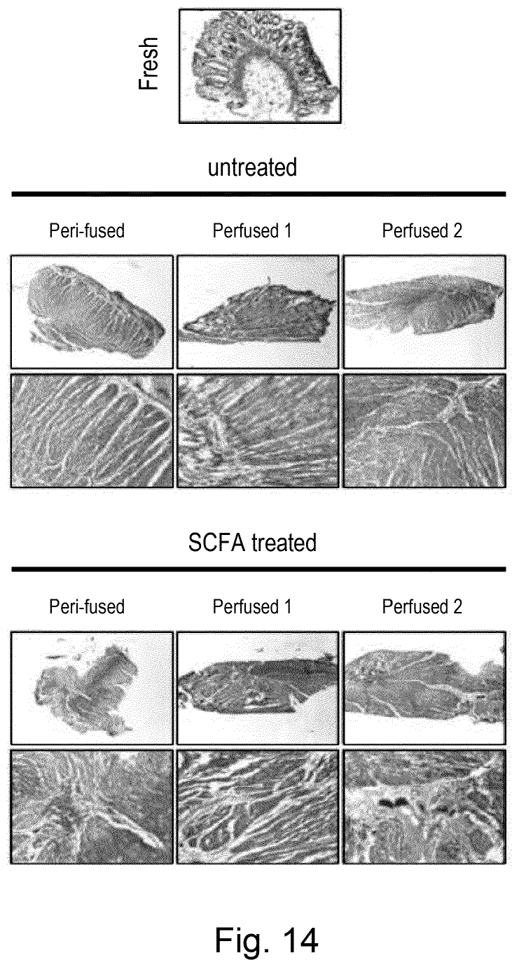

FIG. 15. CONFIGURATIONS FOR DEVICE USE. Use configuration 1 to dispense from syringe A. Use configuration 2 to disconnect syringes A and B. Use configuration 3 to dispense from syringe B. Use configuration 4 to dispense from both syringes A and B.

### DETAILED DESCRIPTION OF THE INVENTION

According to a first aspect, the invention provides a method for perfusing a biological sample in a device comprising a channel comprising a constriction wherein the method comprises flowing a liquid through the channel, sealing the channel with the biological sample and maintaining a flow of liquid within the biological sample.

In the context of the invention, perfusion refers to a flow of liquid through the biological sample. Perfusion comprises both advective and diffusive transport within the tissue. Perfusion should therefore be distinguished from “perifusion”, which refers to a flow of liquid around a biological sample. Perifusion consists of diffusion only.

Diffusion (also referred to herein as “diffusive flow”) refers to passive transport of a substance, for example from the outside of the biological sample to the core of the biological sample. Advection (also referred to herein as “advective flow”) refers to the transport of a substance by the flowing fluid that carries it. In this context, a technical effect of perfusion compared to perifusion is to dramatically increase via advection the available surfaces through which and/or routes by which diffusion may occur. Accordingly, perfusion allows a greater penetration of a substance from the outside of the biological sample into the core of the biological sample. Put another way, a substance exogenous to the biological sample has greater access to the biological sample by the method of the invention. A greater volume of the biological sample may be exposed to the substance by the method of the invention.

The flow is within the biological sample. In other words, liquid may pass through structures within the biological sample. The flow is typically through structures within the biological sample. The flow typically passes through structures within the biological sample. The structures within the biological sample may be endogenous structures. Without being limited by theory, flow within the biological samples may be through the vasculature and/or extracellular space of the biological sample. Accordingly, the structures within the biological sample may be the vasculature and/or the extracellular space. The method does not require cannulation of the biological sample.

The flow of liquid within the biological sample may be an advective flow of liquid within the biological sample. Advective flow is within the biological sample. In other words, liquid may pass through structures within the biological sample. The advective flow is typically through structures within the biological sample. The advective flow typically passes through structures within the biological sample. The structures within the biological sample may be endogenous structures. Without being limited by theory, advective flow within the biological samples may be through the vasculature and/or extracellular space of the biological sample. Accordingly, the structures within the biological sample may be the vasculature and/or the extracellular space. The method does not require cannulation of the biological sample. Indeed, the methods and devices of the invention achieve flow through the specimen instantly, without the need for any other manipulation, such as vascularisation.

Advection within a biological sample allows quicker delivery and critically deeper penetration of oxygen and nutrients into the sample. This in turn contributes towards its longer preservation. In addition to increased biological sample viability ex vivo, this method can be used as a platform for more in vivo relevant studies of responses to a medical intervention. Indeed, advective delivery of pharmaceutical substances in the biological sample's vascular network and core present a more realistic model to simulate drug bioavailability in humans. In most tissue models, pharmaceutical ingredients can take hours to diffuse several cell layers deep within the specimen and may never reach the sample core. On the contrary, in this method, a drug could be administrated to the tissue sample within minutes and a response signal can be measured within a clinically-relevant timeline.

The method comprises “maintaining” a flow of liquid within the biological sample. The skilled person will understand that in the context of the invention, maintaining a flow involves sustaining that flow for a period of time useful for perfusion of a biological sample. In other words, the flow of liquid is not transient. For example, a flow may be maintained for at least around 1 minute, at least around 5 minutes, at least around 10 minutes, at least around 30 minutes, at least around 2 hours, at least around 4 hours, at least around 4.5 hours, at least around 5 hours, at least around 10 hours, at least around 12 hours, at least around 14 hours, at least around 16 hours or at least around 20 hours.

Flow may be maintained for at least around 30 minutes. The flow may be maintained for at least around 0.5 hours, at least around 1 hour, at least around 2 hours, at least around 4 hours, at least around 8 hours, at least around 16 hours, at least around 32 hours, at least around 64 hours, or at least around 128 hours. The skilled person will appreciate that different durations of flow may be adopted for different purposes. For instance, a hydraulic resistance measurement may require only around 30 minutes, or around 2 to around 5 hours. A drug testing experiment may require around 4 or around 4.5 hours, or around 4 to around 16 hours, or around 24 to around 96 hours, or up to 20 days. Since the method is for perfusing a biological sample and the flow must be maintained, the method of the invention does not encompass methods of dissociating a biological sample. The biological sample remains intact during the method. In specific applications the biological sample may be treated with a substance that may result in selective dissociation of a component and/or components of the biological sample. However, the method of the invention does not result in total specimen dissociation.

The flow may be maintained for any suitable duration. The skilled person will appreciate that the duration may be chosen based on the precise purposes of the investigator. For example, the flow may be maintained for up to around 30 minutes, up to around 1 hour, up to around 4 hours, up to around 4.5 hours, up to around 5 hours, up to around 10 hours, up to around 12 hours, up to around 14 hours, up to around 16 hours, up to around 20 hours, up to around 24 hours, up to around 36 hours, up to around 48 hours, up to around 60 hours or up to around 72 hours or up to 20 days.

The transport of solutes within the biological sample (for example the transport of solutes in the fluid within the biological sample) may be quantified by the Peclet number (Pe). In this context, the Peclet number is a dimensionless quantity, defined as the ratio of the rate of advection of a physical quantity by the flow to the rate of diffusion of the same quantity driven by an appropriate gradient. Advection and/or diffusion may be driven by a gradient. A high Peclet number therefore indicates a relatively high contribution of advection to the delivery of liquid within the biological sample (and conversely a relatively low contribution of diffusion). A low Peclet number therefore indicates a relatively low contribution of advection to the delivery of liquid within the biological sample (and conversely a relatively high contribution of diffusion). The flow of liquid within the biological sample may be characterised as having a Peclet number of 1 or more. The Peclet number may be 1.1 or more, 1.2 or more, 1.3 or more, 1.4 or more, 1.5 or more, 1.6 or more, 1.7 or more, 1.8 or more, 1.9 or more, 2 or more, 2.5 or more or 3 or more. The Peclet number may be up to around 100.

The perfusion of a biological sample may be assessed by measuring resistance (difficulty for fluid to flow through the sample). Regardless of how flow is driven, for example whether flow is driven directly by a flow-controlled system or via a pressure-controlled system, any sample presenting a resistance of at least 0.3 mmHg/(μl/min) will have flow of liquid occurring through it. This is based on calculations using Darcy's law, assuming one directional uniform flow through a rigid, cylindrical specimen, minimum dynamic viscosity set to be equal to that of water at 37° C. (7×10\hat{ } (−4)) Pa-s, minimum specimen length in the flow-wise direction set to be 200 micrometers, largest specimen diameter set to be 5 mm and highest conductivity assumed to be equal to that of vitreous humour (3×10\hat{ } (−15) m\hat{ }2). The above calculations result in a resistance of 0.3 mmHg/(μl/min). Therefore, resistance may be at least around 0.3 mmHg/(μl/min). Resistance may be at least around 1 mmHg/(μl/min). Resistance may be selected from the group consisting of at least around 0.3 mmHg/(μl/min), at least around 0.5 mmHg/(μl/min), at least around 0.7 mmHg/(μl/min), at least around 1 mmHg/(μl/min), at least around 1.1 mmHg/(μl/min), at least around 1.2 mmHg/(μl/min), at least around 1.3 mmHg/(μl/min), at least around 1.4 mmHg/(μl/min), at least around 1.5 mmHg/(μl/min) and at least around 2 mmHg/(μl/min). The skilled person will appreciate that resistance may change over perfusion duration due to compression or treatment with a substance. Resistance values given herein may refer to initial resistance, average resistance or maximum resistance. Typically, resistance values given herein refer to average resistance. Resistance of biological samples within then device under perfusion may be up to around 900 mmHg/(μl/min).

In the context of the invention, flow through the biological sample occurs because the channel is sealed with the biological sample. The device may be described as having a fluid path through the channel and through the constriction. The biological sample occludes the fluid path when it seals the channel. The device may therefore be described as configured to maintain a flow of liquid within the biological sample. Because the channel is sealed with the biological sample, the liquid must flow through the biological sample for the flow of liquid to be maintained. The flow of liquid through the biological sample promotes preservation/viability of the sample. The methods of the invention may therefore be considered methods of preserving or maintaining the viability of a biological sample, for example ex vivo.

The biological sample seals the channel without requiring adhesives. Similarly, the biological sample seals the channel due to the pressure gradient in interaction with the device, without requiring modifications to the device dimensions during use. Accordingly, the device may be described as having fixed dimensions. Likewise, the sealing may be described as “self-sealing”, accordingly.

The seal may be quantified by its resistance. In this context, the resistance may be defined how easily fluid flow occurs through the channel. When the biological sample seals the channel, resistance will increase. A high resistance therefore generally indicates a strong seal. A low resistance therefore generally indicates a weak seal. The resistance of the seal may be at least around 1 mmHg/(μl/min). The resistance of the seal may be selected from the group consisting of at least around 0.3 mmHg/(μl/min), at least around 0.5 mmHg/(μl/min), at least around 0.7 mmHg/(μl/min), at least around 1 mmHg/(μl/min), at least around 1.1 mmHg/(μl/min), at least around 1.2 mmHg/(μl/min), at least around 1.3 mmHg/(μl/min), at least around 1.4 mmHg/(μl/min), at least around 1.5 mmHg/(μl/min) and at least around 2 mmHg/(μl/min).

Sealing can be quantified by measuring a non-linear, profound increase in resistance when the sample reaches the constriction. The increase may be for example, around a 100-fold increase. After that, resistance will typically be stable. Resistance may very slowly increase over time due to compression of the biological sample. Resistance as used herein refers to the initial resistance after it increases when the sample reaches the constriction. The skilled person will understand that the hydraulic resistance measurements disclosed herein, such as at least around 1 mmHg/(μl/min), may be generally applicable, but specimen resistance may vary due to the inherent variability of tissue permeability within all organs. For example, two samples may seal equally well the constriction but one may have bigger vessels than the other and therefore present different hydraulic resistance.

The resistance of the seal may alternatively be expressed as a fold increase in resistance after sealing compared to before sealing. The increase may be around a 10-fold increase, around a 100- fold increase, or around a 1000-fold increase.

The biological sample may be any suitable biological sample with a three-dimensional structure. In the context of this invention, individual or dissociated cells are nota suitable biological sample. The biological sample may be a tissue sample, an organoid or sample thereof, a scaffold, a gel, a spheroid, a decellularized tissue specimen, a wafer. The biological sample may be a live ex vivo or in vitro biological sample. The biological sample may remain a live ex vivo or in vitro biological sample for the duration of method. The biological sample may be fixed during the method, for example to aid downstream processing of the biological sample after completion of the method. The method may further comprise a fixing the biological sample, optionally by perfusing the sample with a fixative. The fixative may be any known fixative such as paraformaldehyde, methanol, formalin, ethanol, acetone or osmium. Alternatively, or in addition, the biological sample may be analysed while it remains a live ex vivo or in vitro biological sample. The method may further comprise analysing the biological sample while it seals the channel. Analysing the biological sample may include conducting imaging, hydraulic resistance measurements, biomarker quantification in specimen lysate, real time ELISA, real time PCR, downstream quantification of biomarkers in the perfusate, spectroscopy, ultrasound, x-ray imaging or electrophysiology. Typically, if the method comprises analysing the biological sample while it seals the channel and fixing the biological sample, fixing the biological sample will take place after the biological sample has been analysed while it seals the channel.

After fixation, the biological sample may be subjected to histological analysis. The method may therefore further comprise histological analysis of the fixed biological sample. The histological analysis may be by any known method, such as immunohistochemistry, immunocytochemistry, immunofluorescence, optical microscopy, a common histology stain processing as for hematoxylin and eosin, oil red or Schiff's dye.

The tissue sample may be from an animal or a human. The tissue sample from an animal may be from a laboratory animal such as a model organism. The tissue sample may be from mouse, rat, zebrafish, chicken, pig, primate or dog. The animal or human may be healthy. Alternatively, the animal or human may be diseased. The tissue sample from a human may be from a patient. The tissue sample from a human may be a clinical sample. The tissue sample may come from a donor. The tissue may have been provided after surgery for tissue resection.

The biological sample may be a biopsy. The biological sample may be from tissue resected during surgery. The biological sample may be from any suitable organ. The biological sample may be from any vascularised, poorly vascularised or avascular tissue. The biological sample may be from vascularised tissue. The biological sample may be from ovary, liver, heart, kidney, brain, oesophagus, skin, breast, colon, rectum, lung, prostate, muscle, lymphatics, endothelium, gall bladder, bladder, or pancreas. Typically, the biological sample may be from liver, heart or kidney. The biological sample may be from healthy tissue or from diseased tissue.

The biological sample may be from a tumour. The tumour may be any suitable solid tumour. Accordingly, the biological sample may be from cancerous tissue. The biological sample may be from cancerous tissue selected from the group consisting of ovarian cancer, liver cancer, heart cancer, kidney cancer, brain cancer, oesophageal cancer, melanoma, breast cancer, colorectal cancer, lung cancer, prostate cancer, muscle cancer, lymphoma and pancreatic cancer. The biological sample may be from ovarian cancer.

The biological sample may be of any suitable size. The biological sample may have a longest dimension of at least 0.4 mm. The biological sample may have a longest dimension of at least 0.5 mm. The biological sample may have a longest dimension of at least 1.5 mm. The biological sample may have a longest dimension of up to around 5 mm. The biological sample may have a longest dimension of up to around 20 mm. The longest dimension of the biological sample may be around 1.5 mm to around 5 mm. Typically, the biological sample will have a longest dimension of around 3 mm. The biological sample may have a shortest dimension of at least 0.5 mm. The biological sample may have a shortest dimension of at least 1.5 mm. The biological sample may have a shortest dimension of up to around 5 mm. The biological sample may have a shortest dimension of up to around 20 mm. The shortest dimension of the biological sample may be around 1.5 mm to around 5 mm. Typically, the biological sample will have a shortest dimension of around 3 mm.

The biological sample may be of any suitable volume. The biological sample may have a volume of at least 0.125 μl. The biological sample may have a volume of up to 8 ml. The volume of the biological sample may be around 4 μl. The volume of the biological sample may be around 4 μl to around 50 μl. Typically, the biological sample will have a volume of around 9 μl.

The biological sample may be of any suitable mass. The biological sample may have a mass of at least 3 mg. The biological sample may have a mass of up to 8 g. The mass of the biological sample may be around 6 mg to around 26 mg. Typically, the biological sample will have a mass of around 14 mg.

The biological sample may be obtained by any suitable means. For example, the biological sample may have been obtained by a biopsy performed in vivo. The biological sample may have been obtained by a biopsy performed ex vivo, for example on resected tissue. The biopsy may be obtained using any suitable means, for example a biopsy punch, a Vibratome, a microtome, a biopsy needle, a tru-cut® needle, a lancet, a razor blade. The biological sample may be a 3 mm diameter biopsy of resected liver, optionally mouse liver.

The effects of the invention may be most pronounced for samples above a minimum threshold size. The minimum threshold size may be any suitable size identified herein. For example, the sample may have a longest dimension of at least 50 μm. Preferably, the sample may have a longest dimension of at least 500 μm. Without being bound by theory, when penetration distance is too small (for example, less than 50 μm) diffusion may be faster than advection in the sense that a substance may diffuse more quickly if compared to the time it takes to travel from a given point to another. But rate of diffusion drops off according to 1/L², where L is the characteristic size. Therefore, over large penetration distance and given also consumption by cells, delivery of a substance by diffusion is negligible for longer Ls.

The method of the invention may be an in vitro method or an ex vivo method.

The device comprises a channel comprising a constriction. The channel may be any conduit suitable for providing a liquid flow path within the device. Accordingly, the channel may be of any suitable shape. The channel may have a circular cross section. Alternatively, the channel may have one or more flat surfaces. For example, the channel may have one flat surface. The channel may have three flat surfaces.

The channel may have any suitable dimensions. For example, the channel may have a height of about 0.1 mm to about 50 mm, such as around 1 mm to around 10 mm. The channel may have a width of about 0.1 mm to about 50 mm, such as around 1 mm to around 10 mm. The Height to Width ratio may be around 0.02 to 500, such as around 0.1 to 10. The cross-sectional area of the channel may be around 0.01 mm² to around 25 cm², such as around 0.1 mm² to around 200 mm². One or more dimension of the channel may be consistent along its length. One or more dimension of the channel may vary along its length.

The channel may have dimensions selected from the group consisting of a height of around 1.80 mm, a width of around 1.56 mm, a Height to Width ratio of about 1.14 and a cross-sectional area of about 2.55 mm².

In a specific embodiment, the channel may have a height of around 1.80 mm, a width of around 1.56 mm, a Height to Width ratio of about 1.14 and/or a cross-sectional area of about 2.55 mm².

The constriction may be a narrowing of the channel. The constriction may alternatively be termed a stenosis. The constriction may be a reduction in the width, height and/or cross-sectional area of the channel. The constriction may have the same cross-sectional shape as the channel. Alternatively, the constriction may have a different cross-sectional shape to the channel. The constriction prevents passage of the biological sample through the full length of the channel.

The constriction may have any suitable dimensions. For example, the constriction may have a height of about 0.05 mm to about 49.9 mm, such as around 1 mm to around 9.9 mm. The constriction may have a width of about 0.05 mm to about 49.9 mm, such as around 1 mm to around 9.9 mm. The Height to Width ratio may be around 0.02 to 500, such as around 0.5 to 5. The cross-sectional area of the channel may be around 0.03 mm² to around 24.9 cm², such as around 0.09 mm² to around 150 mm². One or more dimension of the channel may be consistent along its length. One or more dimension of the channel may vary along its length.

In one embodiment, the constriction region may have a width up to around 49.9 mm, a height up to around 49.9 mm, a Height to Width ratio of up to about 50 and/or a cross-sectional area of up to about 2490 mm².

In one embodiment, the constriction region may have a width around 0.65 mm, a height around 0.75 mm, a Height to Width ratio of about 1.15 and/or a cross-sectional area of about 0.44 mm².

The channel may comprise a constriction region. The constriction region may be a longitudinal region of the channel over which the width, height and/or cross-sectional area of the channel changes to form the constriction. The constriction may be the portion of the constriction region having the smallest width, height and/or cross-sectional area.

The constriction region may have length from about 0.1 mm to about 50 mm, such as around 0.5 mm to around 5 mm. The constriction region may have length of about 2 mm. The constriction region length may be greater than 50 mm, for example where one or more dimension of the channel is not consistent along its length, the entire channel from the largest dimension (such as the highest cross-sectional area) to the smallest dimension (such as the smallest cross-sectional area) may be termed a constriction region.

The constriction region may have an angle of about 10 to about 75 degrees to the longitudinal axis of the constriction region. The constriction region may have an angle of about 10 to about 45 degrees to the longitudinal axis of the constriction region. The constriction region may have an angle of about 15 degrees to the longitudinal axis of the constriction region.

The constriction region may have a ratio of maximum to minimum cross-sectional area of at least about 1.05. The constriction region may have a ratio of maximum to minimum cross-sectional area of up to about 20. The constriction region may have a ratio of maximum to minimum cross-sectional area of around 2 to 10. The constriction region may have a ratio of maximum to minimum cross-sectional area of about 5.76.

In one embodiment, the constriction region may have a length of around 2 mm, an angle of around 12.82 degrees to the longitudinal axis of the constriction region and/or a ratio of maximum to minimum cross-sectional area of about 5.76.

The angle of the constriction region to the longitudinal axis of the constriction region may alternatively be expressed as the corresponding angle to a transverse axis of the constriction region. Transverse here means perpendicular to the longitudinal axis. For example, an angle of of around 12.82 degrees to the longitudinal axis of the constriction region corresponds to an angle of around 77.18 to the transverse axis of the constriction region.

The constriction region may comprise a first constriction region portion and a second constriction region portion. The first and second constriction region portions may be separated by the constriction. Likewise, the channel on either side of the constriction region may be termed a first channel portion and a second channel portion, respectively. Therefore, the channel may comprise, in order, a first channel portion, a first constriction portion, a constriction, a second constriction portion and a second channel portion. The constriction is typically in a plane perpendicular to the longitudinal axis of the channel. The constriction may therefore be in a transverse plane with respect to the longitudinal axis of the channel. The first and second constriction region may be symmetrical on either side of the constriction. The first and second constriction region may therefore have transverse symmetry. Alternatively, the first and second constriction region may have transverse asymmetry. The first and second channel region may be symmetrical on either side of the constriction region. The first and second channel region may therefore have transverse symmetry. Alternatively, the first and second channel region may have transverse asymmetry. Without being bound by theory, anisotropy may favour biological sample immobilisation.

In some embodiments, the channel comprises at least one inlet and at least one outlet. Fluid may be flowed through the device, passing first through an inlet and then through an outlet. The constriction is disposed between the inlet and the outlet. Hence the channel may comprise, in order, an inlet, a constriction, and an outlet. The fluid and the biological sample may pass through the same inlet or they may pass through different inlets. Multiple inlets may be useful, for example for flowing different liquids through the device. Suitably, the fluid or fluids must pass through the channel comprising a constriction to reach the biological sample.

In some embodiments, the channel may comprise, in order, an inlet, a first channel portion, a first constriction portion, a constriction, a second constriction portion, a second channel portion and an outlet. The biological sample and the fluid may pass through the same inlet, or they may pass through different inlets.

Because the channel is sealed by the biological sample, the fluid must pass through the biological sample to reach an outlet (e.g. via a second constriction portion and a second channel portion). In this way, perfusion of the biological sample can be achieved instantly.

In some embodiments, the device comprises a first channel portion, a first constriction portion, a constriction, a second constriction portion and a second channel portion, and the biological sample and the fluid pass through the same first channel portion and/or first constriction portion. In some embodiments, the device comprises at least one inlet, a first channel portion, a first constriction portion, a constriction, a second constriction portion and a second channel portion, and the sample and the fluid pass through the same first channel portion and/or first constriction portion, although the sample and the fluid may pass through different inlets.

The device may be made of any suitable material. The skilled person appreciates that the surfaces of the device that interface with the liquid and/or the biological sample are biologically inert. The device may comprise a biologically inert coating on the surfaces of the device that interface with the liquid and/or the biological sample. The device may be formed of a material that is biologically inert. Suitable biologically inert materials include polydimethylsiloxane (PDMS), glass, Polymethyl methacrylate (PMMA), Polyethylene terephthalate (PET), Polycarbonates (PC), Polyimide (PI), silicon, nylon, Polystyrene (PS), Polyethylene glycol diacrylate (PEGDA), Perfluorinated compounds (e.g. Polyfluorinated ethylene propylene (PFEP), Perfluoroalkoxy alkane (PFA), Perfluoropolyether (PFPE), Polyurethane (PU), paper and/or hybrids of these materials. The skilled person will appreciate that any bioinert material that can be machined to have a channel conformation and can be in solid state at room temperature and at 37° C. can be used for fabricating the device.

The device may comprise an inlet reservoir. The inlet reservoir may comprise an opening for insertion of the biological sample. The opening may be any appropriate size for convenient insertion of the biological sample. The inlet reservoir is fluidically connected to the channel. The inlet reservoir may be described as fluidly connected to the channel. The inlet reservoir may therefore allow passage of the biological sample into the channel. The inlet reservoir may therefore allow passage of the biological sample into the first channel portion. The method may further comprise inserting the biological sample into the inlet reservoir of the device, wherein the inlet reservoir is fluidically connected to the channel. Two or more channels may be connected to the same reservoir. The method may comprise flowing the liquid through the inlet reservoir (i.e. the same inlet reservoir into which the biological sample is inserted). Alternatively, the liquid may be flowed through a different inlet, but still through the same first channel portion as used for insertion of the biological sample into the device.

The liquid may be flowing through the channel by any suitable means. For example, the liquid may flow under a pressure gradient. Any pressure gradient could be used to force flow due to the very low resistance presented by the channel alone. The higher the pressure gradient applied the higher the flow rate of the liquid flowing through it. The pressure gradient may be between about 0.5 and about 250 mmHg. The pressure gradient can be measured using a pressure sensor. The pressure sensor may be connected to a computer using a DAQ card. Flow may be driven directly by a flow-controlled system or indirectly via a pressure-controlled system.

In some embodiments, the device of the invention does not comprise additional inlets for flowing liquid through the device or through the biological sample. Instead, the biological sample and the liquid enter the device through the same inlet. The inlet may be the channel inlet and/or the opening of the inlet reservoir. Alternatively, the device of the invention may comprise a plurality of inlets for flowing liquid through the device or through the biological sample. The biological sample and the liquid may enter the device through the same inlet or through different inlets

The pressure gradient may be generated by a pump. The pump may be a syringe pump.

Tubing may be connected at a device outlet. The tubing may be connected to a syringe, such as a plastic syringe, which is connected to the syringe pump. The main body of the syringe may be immobilised by a syringe holder of the syringe pump, whilst the end of the syringe is connected to the moving part of the pump. In this configuration, flow through the device is controlled by withdrawing media.

Alternatively, the tubing may be connected to a device inlet. In this configuration, the syringe pump would work by infusing media.

Both the inlet and the outlet may be connected to tubing, for instance without a reservoir. In this configuration, pump function could be either set to withdraw or dispense media, depending on the user's preference. In the case of no reservoir, tubing diameter should be assessed with respect to biological sample dimensions so that it can still be incorporated into the device and directed to the constriction.

The flow rate may be any rate suitable to cause the biological sample to move through the channel towards the constriction so that it can seal the channel. The flow rate should not be so high that the biological sample is forced through the constriction so that it can no longer seal the channel. The flow rate may be at least about 10 nanolitres/min. The flow rate may be less than about 1 millilitre/min. The flow rate may be between about 10 nanolitres/min and about 1 millilitre/min. The flow rate may be between about 40 nanolitres/min and about 250 nanolitres/min. In one embodiment, the flow rate may be around 200 nl/min. In a preferred embodiment, the flow rate may be around 100 nl/min.

The skilled person will appreciate that the seal may occur at the constriction and/or upstream of the constriction in the flow path, such as in the first constriction portion and/or in the first channel.

The device may comprise parallel channels. The parallel channels may allow testing of multiple samples in parallel and/or having several replicates of the same condition. Typically, one sample is used per channel, so the number of samples tested will typically equal the number of channels. The device may comprise two or more parallel channels. The device may comprise up to around 100 parallel channels. The skilled person is aware how to modify the flow rate and/or pressure gradient and associated equipment to accommodate parallel channels. For a pressure-controlled configuration the skilled person will appreciate no changes to flow rate and/or pressure gradient are required to incorporate parallel channels. For a flow-controlled configuration the skilled person is aware how to modify the flow rate and/or pressure gradient and associated equipment to accommodate parallel channels. If the flow in each channel is controlled by a syringe (for example during perfusion) then no modification should be needed due to the presence of parallel channels.

However, each syringe pump can control up to a certain number of syringes, depending on its structure. The skilled person is able to make small modifications to connect the parallel channels to manifolds or to use an adapter for the syringe pump, which would allow more syringes to be controlled in parallel. If all parallel channels are controlled by the same syringe, then the flow rate controlled by the syringe pump has to be adjusted based on the following formula:

(Final flow rate)=(number of devices or channels)×(flow rate for one device or channel)

Alternatively, the method may involve the use of multiple devices in parallel. The devices may be single channel devices or multi-channel devices.

The method may comprise perfusing two or more biological samples in parallel. The two or more biological samples may be from the same patient and/or from the same animal. Alternatively, the two or more biological samples may be from different patients and/or animals.

The liquid may be any suitable liquid for maintenance of the biological sample. The skilled person is aware of suitable liquids for maintenance of biological samples. For example, the liquid may be saline, phosphate buffered saline (PBS), fixative, or tissue culture medium. The liquid may comprise antibiotics, hormones, proteins, drugs, preservatives, fixatives, pH indicators, pH buffers, essential and/or non-essential amino acids, dyes, sugars, alternative carbon sources, fluorescent conjugates of the substances mentioned above and/or x-ray contrast agents.

The liquid could be changed one or more times during the method. The method may further comprise changing the liquid. For example, the method may comprise perfusing the sample only with saline and clot busters for an hour, then switching to media, then after 24 or 48 hours (for instance at the end of the experiment) switching to PBS for an hour and then to fixative (e.g. 4% PFA for an hour). Another example is perfusing the biological samples for 6 hours with media, then switching to media with a drug (e.g. Acetaminophen) and then switching after 2 hours to an antidote for drug's effect (e.g. Acetylcysteine).

The skilled person will appreciate that any suitable drug may be used, including but not limited to Acetaminophen, N-acetylcysteine, Cisplatin and Taxol.

The duration of perfusion of a biological sample may be selected as appropriate. For example, the sample may be perfused from between about two to about 72 hours. In one embodiment, the sample may be perfused for up to 48 hours.

The liquid may comprise a substance of interest. The substance may be dissolved or suspended in the liquid. The substance may be selected from the group consisting of a dye, a bead, a nanoparticle, a liposome, a cell suspension, a virus, a microbe, a peptide or protein, a chemical, DNA, RNA, a plasmid, a transcription factor and a gene editing construct. The bead may be a polystyrene bead. The substance may be a pharmaceutical substance. The pharmaceutical substance may be selected from the group consisting of a small molecule, a biological molecule and a cell. The small molecule may be a chemotherapeutic, such as cisplatin, Taxol, carboplatin, a Poly (ADP-ribose) polymerase (PARP) inhibitor, an angiogenesis inhibitor or an Epidermal growth factor receptor (EGFR) inhibitor. The biological molecule may be an antibody, such as a therapeutic antibody. The cell may be a cell therapy.

The invention allows testing of different dosages of a substance against a given phenotype and/or genotype. The method may comprise use of two or more parallel channels each comprising a sample from the same subject. In this way genotype is controlled. The samples from the same subject may be from the same tissue and or the same sample. In this way phenotype is controlled. The two or more parallel channels may comprise different doses of the same substance. Accordingly, each sample may be exposed to a different dose of the same substance. The method may further comprise analysing the effects of the substance at different doses. The method may further comprise determining the most effective dose.

In operation, the device facilitates tissue specimen loading, immobilisation and perfusion ex vivo. Tissue samples can be loaded in an inlet reservoir and directed to the constriction site by flow controlled with a syringe pump or by manual control of a syringe. This enables the sample to seal the constriction region, restricting flow around the sample. A pressure gradient is then generated across the tissue sample, in order to drive flow through the sample. Effectively, the device enables preservation of tissue viability by advective mass transport through the tissue.

In a highly specific embodiment, the constriction region may have a width around 0.65 mm, a height around 0.75 mm, a Height to Width ratio of about 1.15, a cross-sectional area of about 0.44 mm², a length of around 2 mm, an angle of around 12.82 degrees to the longitudinal axis of the constriction region and/or a ratio of maximum to minimum cross-sectional area of about 5.76. This constriction design has been optimised to facilitate flow-controlled perfusion of 3 mm tissue samples for up to 48 hours. Smaller constrictions result in more efficient immobilisation whilst increasing the hydraulic resistance of the sample in the constriction due to compression. Similarly, larger constrictions result in lower pressure gradient across the sample, while immobilisation becomes less effective. The optimal design addresses these competing effects, allowing efficient specimen immobilisation and perfusion without any treatment of the device surface or sample processing.

According to a second aspect, the invention provides a method for assaying a substance comprising maintaining a flow of liquid within a biological sample by a method according to the first aspect of the invention, wherein the liquid comprises the substance and detecting one or more effects of the substance on the biological sample.

In the context of this invention, “assaying” refers to determining or detecting one or more effects of a substance. The substance is typically a pharmaceutical substance.

The one or more effects may be selected from, but are not limited to hydraulic resistance, apoptosis induction, lipid content depletion, necrosis, starvation, response to hypoxia, liver stellate cell activation, endothelial cell marker loss, antioxidant loss, DNA fragmentation, fibrosis and steatosis.

Detecting may be performed in real time or at an end-point. Detecting in real time may be for example by using imaging. Imaging may be widefield, confocal, two-photon, multiphoton, Raman and/or x-ray imaging to monitor any of the substances/constructs that are mentioned above as potentially mixed with the flowing fluid, their delivery within the tissue and/or, track their movement through vessels or any other porous tissue structure, monitor their accumulation and/or consumption by the cells. End-point detection may be, for example by histology, immunostaining or lysis. Histology may comprise fixed or unfixed tissue for frozen sectioning or paraffin-embedded tissue sectioning. Histology may comprise use of histology dyes including but not limited to Hematoxylin, Eosin, Oil Red O, Schiff's dye, Masson's trichrome and DAB. Immunostaining may comprise colorimetric or fluorometric detection of one or more antigens. Biological samples may be lysed after the experiment for enzyme or gas quantification or to measure the levels of a given metabolic product, drug or reagent.

Detecting may be performed with reference to or by comparison with one or more controls. The control may be a positive and/or a negative control. Detecting may be performed with reference to or by comparison with a positive and/or a negative control. The control may be a parallel assay of a different substance or a liquid with no substance added, with a biological sample from the same source. This allows the genotype and/or phenotype of the sample to be controlled while the effects of one or more substances are assayed. The biological sample from the same source may be a biological sample from the same patient or animal. The biological sample from the same source may be a biological sample from the same tissue. The biological sample from the same source may be a biological sample from the same tissue of the same patient or animal. The biological sample from the same source may be a biological sample from the same biopsy of the same tissue of the same patient or animal. The control may be a parallel assay of a different biological sample with the same substance. This allows the effects of the substance to be controlled while the genotype and/or phenotype of the sample are assayed.

The invention also provides a device for use according to the method of the first and/or second aspect of the invention.

According to a third aspect, the invention provides a device for use in perfusing a biological sample, wherein the device comprises a channel comprising a constriction wherein the constriction has a width of up to around 49.9 mm, a height up to around 49.9 mm, a Height to Width ratio of up to about 50 and/or a cross-sectional area of up to about 2490 mm².

The constriction may be configured to seal the channel with the biological sample in use and to maintain a flow of liquid within the biological sample.

The constriction may have a width of up to around 49.9 mm. The constriction may have a height up to around 49.9 mm. The constriction may have a Height to Width ratio of up to about 50. The constriction may have a cross-sectional area of up to about 2490 mm². The constriction may have a width of up to around 0.65 mm, a height around 0.75 mm, a Height to Width ratio of about 1.15 and/or a cross-sectional area of about 0.44 mm².

The constriction may have a width of up to around 0.65 mm. The constriction may have a height around 0.75 mm. The constriction may have a Height to Width ratio of about 1.15. The constriction may have a cross-sectional area of about 0.44 mm².

According to a fourth aspect, the invention provides a method of manufacturing a device according to the third aspect of the invention, the method comprising bonding a flat surface of a first layer to a flat surface of a second layer, wherein the flat surface of the second layer comprises a groove, wherein the channel is formed by the groove and the flat surface of the first layer.

The method may further comprise bonding a third layer comprising a reservoir to the second and/or first layer, wherein the reservoir is configured to interface with the channel.

The bonding may be by plasma treatment.

Applications, uses and advantages of the invention are further described below.

Liver is the main organ for drug metabolism, however predicting its response to an external stimulus and disease initiation and progression are challenging. To study drug effects and disease biology researchers use animal models and complex cell-based platforms. However, due to evolutionary processes animals poorly mimic human liver physiology, whilst cells quickly lose their characteristic properties and shape in vitro.

Limited understanding of liver injury and disease influences medical intervention effectiveness for patients. Drug induced liver injury (DILI) has been the most frequent single cause of safety-related drug withdrawal for the past 50 years and remains the prime reason for acute liver failure in the USA. In vitro platforms for drug safety assessment are considered as promising tools in drug toxicity with the in vitro toxicology market being expected to worth $12.7 Billion by 2024.

Apart from DILI, several other conditions that progressively compromise liver health remain poorly treated due to lack of suitable medical interventions. Non-alcoholic fatty liver disease (NAFLD), Non-alcoholic steatohepatitis (NASH) and viral hepatitis (VI) are complex conditions that are poorly recreated in vitro and as treatment means are limited liver disease is rendered as a clinical priority. Global liver diseases therapeutics represent a market that is expected to worth about $19.6 Billion by 2022.

Tissue samples comprise most cell populations present physiologically within organs. A device that preserves thick liver specimens for up to 48 hours via perfusion may be applicable in a plethora of settings where tissue response to a drug is of interest. Perfused liver specimens may be used to assess how cell type-specific stimuli may affect a population of cells versus another and the tissue. The current device development status allows device use for applications focusing on cell damaging effects that may be induced by a drug, working as a higher relevance in vitro tool. Pro-apoptotic and -necrotic and events can be analysed with endpoint assays and compared to perfused controls, which maintain high viability.

Liver specimen perfusion within this device has preserved cell lipid content to physiological levels. As several drugs cause liver damage via accumulation of lipid droplets within cells (steatosis), this device would be applicable as a screening tool to identify early compounds with steatotic effects in the drug discovery pipeline. Similarly, as specimens are perfused through their vasculature, effects of vasculature-targeting drugs could be assessed. Also, the current set up enables hydraulic resistance measurements with thick liver samples. As profibrotic and stiffening processes increase tissue hydraulic resistance, this platform may be used to evaluate an intervention's influence on tissue mechanical properties.

In the case of perfused liver specimens, services that can be currently performed with this technology may involve apoptosis, necrosis and steatosis screening and tissue property alteration.

Every year over 360,000 people are diagnosed with cancer in the UK. To treat these patients, doctors use a “trial and error approach” when prescribing cancer drugs, which often result in increased side- effects and ineffective therapy. Doctors take into consideration factors such as age, overall health, type and stage of cancer, and medical history to then refer to the literature before prescribing treatment. Although it is acknowledged that treatment should be selected based on officially established guidelines, there actually is no single authoritative source for chemotherapy regimens. Clinicians usually start by prescribing standard doses and then see how a patient reacts which often leads to ineffective treatment and unwanted dangerous side-effects.

The invention allows the clinician to shorten that “trial and error” process and thus developed a more personal approach to the management of cancer. By utilizing the patient's own tissue biopsy the invention has the ability to determine drug toxicity outside of the patient's body. This technology will be able to test all cancerous parenchymal (solid) tissue. In other words, it will provide a personalized analysis of treatment to cancer patients with cancers such as; lung, liver, breast, cervical, ovarian, pancreatic, and kidney among others. A priority is ovarian cancer, which was determined by two factors. First, ovarian cancer provides an abundance of available tissue for testing due to the use of debulking surgery by physicians as a first line of treatment 80% of the time. Second, because ovarian cancer presents an emergent clinical need that urgently needs to be addressed.

Approximately 75% of women who are diagnosed with ovarian cancer are already at advanced stages II-IV. Very often being treated with ineffective drugs results in a recurrence of the cancer. In fact, it is estimated that 80% of women diagnosed with advanced stages of ovarian cancer will have recurrence of their disease due to ineffective treatment. It has become clear that the traditional “one size fits all” method of therapy is inefficient when considering the quality of treatment for patients. Studies have shown that over 50% of women 65 and over, with advanced ovarian cancer, receive inadequate treatment regimens.

Personalized treatments have not yet been standardized completely when managing cancer, and so many patients are either under-treated or over-treated. While being under-treated reduces side-effects and costs, it often leads to rapid progression of the disease. On the other hand, being over-treated, which usually has a higher chance of working, increases healthcare costs and exposes patients to life-threatening side-effects. Sources have shown that, on average, 75% of cancer patients receive ineffective treatment. This emphasizes the huge impact a more personalized and precise treatment could have on a patient's health, their quality of life, and our healthcare system.

Currently, nearly all patients diagnosed with any form of ovarian cancer will receive surgery, known as debulking surgery, as the primary line of treatment. According to the National Comprehensive Cancer Network (NCCN) guidelines, this surgery is to both assess the extent of the cancer and to remove as much of it as possible from the body. Once the procedure has been performed, patients are then treated with different therapy regimens depending on the stage of their cancer and their overall health. A total of 19 chemotherapy drugs are currently in use against ovarian cancer.

The small number of women diagnosed at stage I ovarian cancer usually receive debulking surgery but do not go on to be treated with chemotherapy. However, all women diagnosed with stages II-IV are most commonly treated with a combination of a platinum compound (usually cisplatin or carboplatin) with a taxane (usually paclitaxel or docetaxel) for about 3-6 cycles after their primary surgery. This has been the defined standard of care for ovarian cancer for a long time and is based on various clinical trials. Unfortunately, the lack of randomized data in every type of clinical setting has forced oncologists to make inferences on the treatment they prescribe.

Once patients have been appropriately staged and optimally or sub-optimally debulked they will decide with their clinician the treatment plan. It is important to note that patients described as “platinum-sensitive” are those who have had a treatment free interval for longer than 6 months after their first-line of treatment, while those who are treatment free for less than 6 months are described as “platinum-resistant”.

The device of the invention is a microfluidic device that may be used to screen patients, with recurring or stage II-IV ovarian cancer, against a range of cancer therapies to provide the most effective treatment possible. Unlike other devices of its kind, this device can provide results between 24-48 hours. The same tumor tissue retracted during debulking surgery, which as stated earlier the vast majority of patients receive, can be used to be tested in the device. This avoids disrupting the clinician's workflow to help in avoiding ineffective treatment and unwanted side-effects before prescribing treatment post-surgery.

Competitive advantages of the device of the invention include:
Current direct competitors each only test for the indication of one ovarian cancer drug. This device has the potential to give indications for the use of up to ten therapies.
This device can use therapies that have already been approved, thus opting for a simpler regulatory pathway.
All current technologies for companion diagnostics use Immunohistochemistry, In Situ Hybridization, Next Generation Sequencing, or Polymerase Chain Reaction techniques. This device can use perfusion and a cell viability assay.
This product can be widely commercialised. Surgical theatres or pathology labs are two potential users. Results can be read between 24-48 hours, which may assist decreasing waiting time for the patient and improving survival rates.
The majority of women are already receiving surgery as a primary form of treatment; therefore, this device will not disrupt the workflow of the oncologist.

Preferred features for the second and subsequent aspects of the invention are as for the first aspect of the invention mutatis mutandis.

The present invention will now be described by way of reference to the following Examples and accompanying Drawings which are present for the purposes of illustration only and are not to be construed as being limiting on the invention.

### EXAMPLE 1

### Fabrication Protocol

A. The device comprises three layers:
1) Reservoir: Reservoirs are fabricated using a mould, where a mixture of Sylgard 1 8 (formulated product for PDMS fabrication comprising elastomer, catalyst and clearing agents) and curing agent (10:1) is cast in it. After filling the mould with the mixture, the construct is placed within a desiccator and the mixture is degassed using a common air pump for 45′. Next, PDMS is cured at 65° C. for at least 6 h.
2) Channel layer: Device main layer with channel architecture is fabricated using a (different) mould, following the same steps as for reservoir fabrication.
3) Bottom layer: A flat layer of the same material and processing is used as the bottom of the device. Usually it is fabricated using a Petri dish.

B. To form the final device the three layers are bonded one on top of the other using plasma treatment.
a. First, the bottom layer (preferably the surface that was in direct contact with the Petri dish during fabrication) and the surface of the channel layer that was in direct contact with the mould are treated for 45″ with a plasma wand.
b. After that, the two layers are interfaced, manually compressed against each other and then (whilst remaining attached) are cured on a hot plate, at 80° C. for 3 h.
c. Similarly, the reservoir is bonded on top of the channel layer opening using plasma treatment (same process as 4.). The three-layer construct is let to cure on a hot plate, at 80° C. for 5 h.

C. Tubing connection: It is recommended that autoclavable tubing is used so that device sterilisation can be performed in full via autoclave treatment. Indicative tubing length is 80 cm and tubing outer diameter is 1.06 mm.
a. Using a common biopsy punch, a cylindrical domain is cut out of a flat PDMS layer (similar to the one on 3.).
b. Similarly, a circular domain that matches tubing's outer diameter (±0.1 mm) is extruded out using a biopsy punch. Each donut-like part is connected to each end of the tubing.
c. One end of the tubing is interfaced with the device and sealed with 30 μl of uncured PDMS mixture (10:1). The other end is connected to a connector so that is later interfaced with a syringe.
d. The assembled parts are let to cure on a hot plate, at 80° C. for 5 h (until PDMS sealing tubing connections has fully polymerised).

### EXAMPLE 2

### Perfusion Protocol

Any device, device container, piece of tubing, syringe or reagent that will be used has to be sterile. Any Device fabricated through the fabrication protocol found above can be sterilised with standard autoclave treatment (15 min, 121° C.). For heat resistant tubing autoclave sterilisation is preferred. All liquid reagents to be used will be sterile filtered with a sterile 0.2 pm pore filter.
All steps described below must be followed within a laminar flow biological cabinet to protect equipment and reagent sterility.
All parts should be removed from the autoclave/plastic bags and devices should be placed within device containers, with the adapters, the tubing and the three-way valves being connected to the devices.
Recommended equipment for perfusion: syringe A: 1m1; syringe B: 5m1; tubing length: 90 cm.

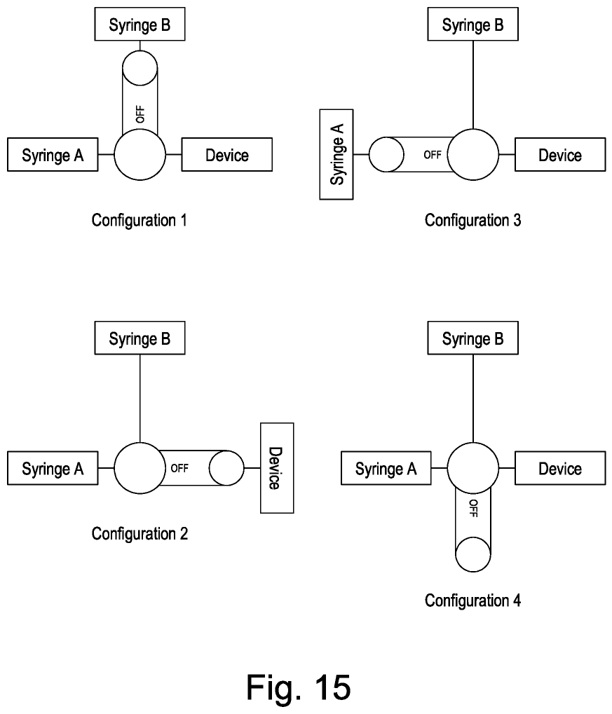

A. Setting up the devices—the configurations referred to below are illustrated in FIG. 15:
1. Fill syringe A (parallel to device tubing; volume depends on syringe pump allowance) and syringe B (perpendicular to device tubing plane; volume depends on total dead volume within device and tubing) with Phosphate Buffered Saline (PBS).
2. Connect each device tubing to a four-way adapter and then connect each adapter to one syringe A and one syringe B. Using configuration 4 dispense enough volume from both so that:
   The device is filled with media up to reservoir edge.
   All dead volume is filled with saline—no air bubbles are observed within the device or tubing.
3. Using a pipette empty the reservoir.
4. Using adapter configuration 1 empty syringe A from saline. Using configuration 3 empty syringe B from saline.
5. Using configuration 2 disconnect syringes A and B. Fill syringes A and B with suitable tissue culture media (depending tissue type and condition) and reconnect them to the adapter as on step 2.
6. Using configuration 4 dispense the whole volume of both syringes.
7. Using a pipette empty the reservoir.
8. Repeat step 5.
9. Using configuration 1 dispense half of the volume within syringe A. Repeat for syringe B using configuration 3. At this point the adapter, the tubing connecting the adapter with the device and the device (up to reservoir edge) should be filled with tissue culture media. No air bubbles should be observed.
   The setup is ready for biological sample incorporation.

B. Biological sample incorporation:
1. Using sterile tweezers collect one biological sample unit (e.g. 3-mm murine liver specimen) and place it within the tissue culture media volume in the reservoir so that is submerged. Wait until it sinks to reservoir bottom.
2. Change adapter configuration to configuration 3 and using syringe B induce momentum perturbations (withdraw and dispense) so that the specimen moves within the liquid volume in the reservoir.
3. Once the biological sample is no longer in contact with device bottom, using syringe B withdraw culture media steadily aiming to direct the biological sample to the inlet of the straight channel of the device. This step may need to be repeated several times to successfully direct the specimen in the channel. When syringe B is full and the specimen is still in the reservoir dispense % of syringe B volume and repeat step 3.
4. Once the biological sample reaches channel inlet continue withdrawing media using syringe B, however at a slower rate, forcing the sample to move with no more than 4 mm/s. Withdraw enough volume so that the specimen is directed within the constriction region of the device.
5. Once the specimen macroscopically appears as it seals the constriction region and is immobile stop any flow perturbation and change adapter configuration to configuration 2.
   The setup is ready for perfusion initiation.

### EXAMPLE 3

### Perfusion Initiation

1) Place all device containers with devices within a CO₂ incubator.
2) Connect syringe A main body to the pump's syringe holder and syringe A end to the moving part of the syringe pump. Configuration 2 allows volume exchange between syringe A and B without the specimen being affected.
3) Once all syringes are connected to the syringe pump, using configuration 1, initiate syringe pump function. (Recommended setting: perfusion at 100 nl/min, withdraw only, no target volume set).

### EXAMPLE 4

### Perfusion End

1) Stop syringe pump function.
2) Change valve configuration to configuration 2. Disconnect each four-way valve from the syringe A it is attached to.
3) Remove device containers that carry the devices out of the CO₂ incubator and place them immediately within the laminar flow biological cabinet.
4) Disconnect syringe B.

Next steps and biological sample treatment depend on the assays used with the sample.

### EXAMPLE 5

### Biological Sample Isolation/Preparation

1. Spray original biological sample container with 70% ethanol and place it in a biological laminar flow cabinet.
2. Remove container lid and using sterile tweezers transfer the biological sample to a sterile petri dish.
3. Wash the biological sample twice with ice cold buffered saline with glucose (recommended glucose concentration 2 g/I).
4. If the biological sample is a tissue specimen divide it in tissue specimens using a sterile biopsy punch. Specimen dimensions are defined by device dimensions. For a given device design tissue samples should be about 3-mm.
5. Wash the specimens twice with ice-cold saline and then transfer them in a sterile container with 5 ml of tissue culture medium.
Biological samples are ready to be incorporated in the device.

### EXAMPLE 6

### Tissue Specimen Loading, Immobilisation and Perfusion Ex Vivo

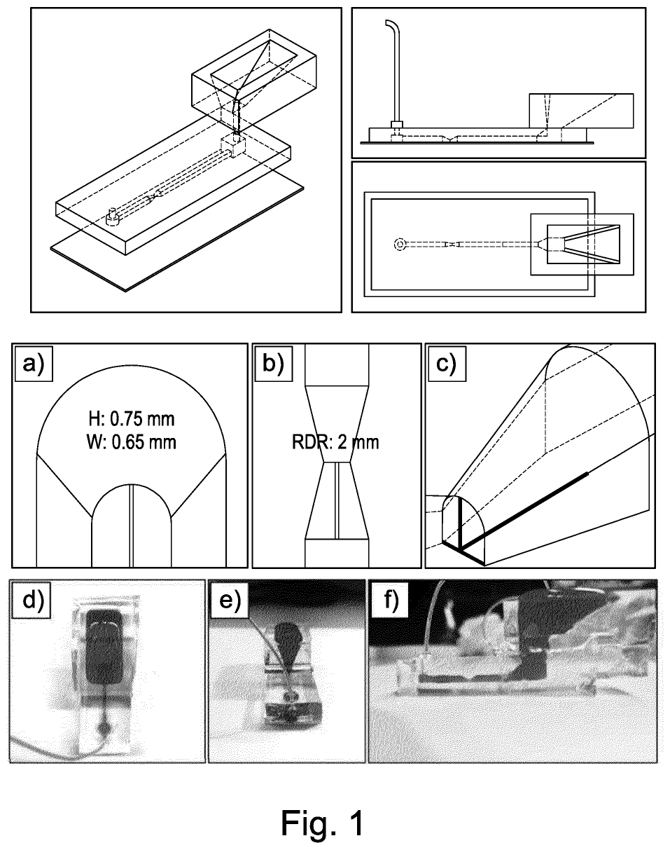

The channel-based device with a special constriction design illustrated in FIG. 1 has been successful in facilitating tissue specimen loading, immobilisation and perfusion ex vivo. Tissue samples can be loaded in an inlet reservoir and directed to the constriction site by flow controlled with a syringe pump. This enables the sample to seal up the constriction region, restricting flow around the sample. A pressure gradient is then generated across the tissue sample, in order to drive flow through the sample. Effectively, the device enables preservation of tissue viability by advective mass transport through the tissue.

The constriction design has been optimised to facilitate flow-controlled perfusion of 3-mm tissue samples for up to 48 hours. Smaller stenosis dimensions result in more efficient immobilisation whilst increasing the hydraulic resistance of the sample in the constriction due to compression. Similarly, larger constriction size favours lower pressure drop, while immobilisation becomes less effective. The optimal design addresses these competing effects, allowing efficient specimen immobilisation and perfusion without any treatment of the device surface or sample processing.

Intra-specimen perfusion has been characterised with a polydimethylsiloxane (PDMS)-based device, where 3-mm murine liver samples were perfused at 200 nl/min for 2.5 hours. Perfusion duration was decided on the basis of allowing enough time for specimen acclimatisation and effective constriction sealing while tracer diffusive transport remaining negligible. To demonstrate perfusion efficacy, tissue samples were perfused with saline containing fluorescent tracers that were afterwards embedded in optimal cutting temperature (OCT) compound and cut into 10-pm thick sections with a cryostat. Static controls were incubated in the same saline-tracer mixture, processed in OCT and cut in sections. Fluorescent tracers were found within the core of perfused specimens while they were practically absent in static controls. Notably, perfusion was mainly through the vasculature as was demonstrated by CD31 staining on cryosections.

The developed platform was used to preserve thick tissue samples ex vivo for long enough to be used for phenotypically-relevant drug response studies. In this context, murine liver samples were cultured in perfused and static (control) conditions for 48 hours. Lactate dehydrogenase (LDH) and Adenosine triphosphate (ATP) quantification in specimen lysates suggest better preservation of cell viability in the perfused device. To account for differences between the employed tissue samples, LDH and ATP data were normalised with total protein. Viability data after two-day culture experiments appear to be paired.

### EXAMPLE 7

### Drug Response Studies

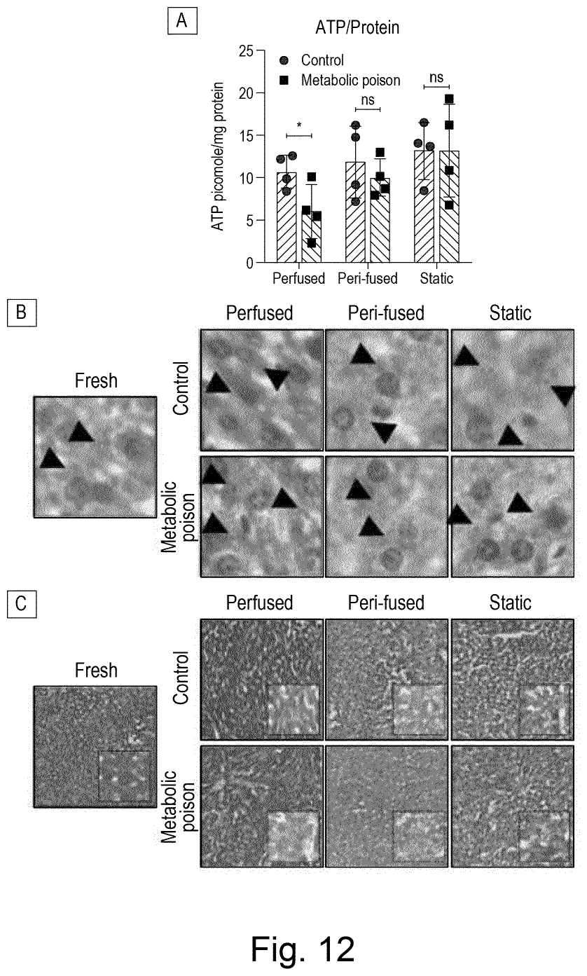

Improved viability maintenance and circulation-mimicking perfusion meansthis system can be used in drug response studies. For example, mouse liver specimens treated with a metabolic poison in this device (see FIG. 12) produced a significant decline in their ATP levels, and lower carbohydrate content and bigger lipid droplets (indicative of stress).

The device has potential in measurement of the cytotoxic effect of a chemotherapeutic. To test this hypothesis specimens from several subjects will be collected, loaded and perfused in the device with a drug to compare endpoint viability between treated samples and non-treated controls. Moreover, to explore this device's potential in the clinical setting, perfusion and viability preservation will be examined with human tissue. More specifically, ovarian tumour samples will be loaded in a parallel- channel platform to validate the system with specimens of human origin. Resected tissue from five patients with naive ovarian tumours will be divided in 3 mm-thick specimens and then loaded, immobilised and perfused within the device. After a 48-hour perfusion, specimens will be recollected from the device and used for lactate dehydrogenase quantification after lysis or cleaved-Caspase 3 and TUNEL staining (cryosections). The data from this experimental design will be used to confirm that this device can be used for in vitro personalised drug response studies. Tissue specimens from five patients will be perfused for 24 hours within the device; half of the specimens will be exposed to a wide-range chemotherapeutic (e.g. cisplatin) while the other half will be treated with a vehicle control. Viability and cytotoxicity data from the two groups will be compared to evaluate whether this assay could be predictive of the potential benefit of a drug for a patient.

1. A method for perfusing a biological sample in a device comprising a channel comprising a constriction wherein the method comprises flowing a liquid through the channel, sealing the channel with the biological sample and maintaining a flow of liquid within the biological sample.

2. The method of claim 1 wherein the flow within the biological sample passes through the vasculature and/or the extracellular space.

3. The method of any preceding claim wherein the flow is maintained for at least around 30 minutes, at least around 2 hours, at least around 4 hours, at least around 4.5 hours, at least around 5 hours, at least around 10 hours, at least around 12 hours, at least around 14 hours, at least around 16 hours or at least around 20 hours.

4. The method of any preceding claim wherein the transport of solute(s) within the biological sample is characterised as having a Peclet number of 1 or more.

5. The method of any preceding claim wherein the resistance of the seal is at least around 0.3 mmHg/(μl/min), at least around 0.5 mmHg/(μl/min), at least around 0.7 mmHg/(μl/min), at least around 1 mmHg/(μl/min), at least around 1.1 mmHg/(μl/min), at least around 1.2 mmHg/(μl/min), at least around 1.3 mmHg/(μl/min), at least around 1.4 mmHg/(μl/min), at least around 1.5 mmHg/(μl/min) or at least around 2 mmHg/(μl/min).

6. The method of any preceding claim wherein the biological sample is a tissue sample, an organoid or sample thereof, a scaffold, a gel, a spheroid, a decellularized tissue specimen ora wafer.

7. The method of any preceding claim, wherein the biological sample is a tissue sample.

8. The method of any preceding claim wherein the biological sample is from liver, ovary, colon, skeletal muscle, heart or kidney.

9. The method of any preceding claim wherein the biological sample is from cancerous tissue selected from the group consisting of ovarian cancer, liver cancer, heart cancer, kidney cancer, brain cancer, oesophageal cancer, melanoma, breast cancer, colorectal cancer, lung cancer, prostate cancer, muscle cancer, lymphoma and pancreatic cancer.

10. The method of any preceding claim wherein the biological sample has a longest dimension of at least 0.4 mm, optionally wherein the longest dimension of the biological sample is around 1.5 mm to around 5 mm, optionally wherein the biological sample has a longest dimension of around 3 mm.

11. The method of any preceding claim further comprising inserting the biological sample into an inlet reservoir, wherein the inlet reservoir is fluidically connected to the channel.

12. The method of any preceding claim wherein the rate of flow is between about 10 nanolitres/min and about 1 millilitre/min, optionally between about 40 nanolitres/min and about 250 nanolitres/min.

13. The method of claim 12 wherein the rate of flow is around 200 nl/min.

14. The method of claim 12 wherein the rate of flow is around 100 nl/min.

15. The method of any preceding claim wherein the constriction has a width of up to around 49.9 mm, a height up to around 49.9 mm, a Height to Width ratio of up to about 50 and/or a cross-sectional area of up to about 2490 mm².

16. A method for assaying a substance comprising maintaining a flow of liquid within a biological sample by a method according to any preceding claim, wherein the liquid comprises the substance and detecting one or more effects of the substance on the biological sample.

17. The method of claim 16 wherein the substance is a pharmaceutical substance selected from the group consisting of a small molecule, a biological molecule and a cell.

18. The method of any one of claim 16 or 17 wherein the effect is selected from the group consisting of hydraulic resistance, apoptosis induction, lipid content depletion, necrosis, starvation, response to hypoxia, liver stellate cell activation, endothelial cell marker loss, antioxidant loss, DNA fragmentation, fibrosis and steatosis.

19. The method of any one of claims 16 to 18 wherein the detecting is performed:
(a) in real time, optionally by imaging; or
(b) at an end-point, optionally by histology, immunostaining or lysis.

20. A device for use in perfusing a biological sample, wherein the device comprises a channel comprising a constriction wherein the constriction has a width of up to around 49.9 mm, a height up to around 49.9 mm, a Height to Width ratio of up to about 50 and/or a cross-sectional area of up to about 2490 mm².

21. The method of any one of claims 1 to 19 or the device of claim 20, wherein the constriction has a width of up to around 0.65 mm, a height around 0.75 mm, a Height to Width ratio of about 1.15 and/or a cross-sectional area of about 0.44 mm².

22. The method of any one of claims 1 to 19 or the device of any one of claim 20 or 21, wherein the channel comprises a constriction region having:
(a) a length from about 0.1 mm to about 50 mm, optionally about 2 mm; and/or
(b) an angle of about 10 to about 75 degrees to the longitudinal axis of the constriction region, optionally about 15 degrees; and/or
(c) a ratio of maximum to minimum cross-sectional area of at least about 1.05, optionally at least about 20, optionally about 5.76.

23. The method of any one of claims 1 to 19 or the device of any one of claims 20 to 22, wherein the constriction region has a length of around 2 mm, an angle of around 12.82 degrees to the longitudinal axis of the constriction region and/or a ratio of maximum to minimum cross-sectional area of about 5.76.

24. A method of manufacturing a device according to any one of claims 20 to 23, the method comprising bonding a flat surface of a first layer to a flat surface of a second layer, wherein the flat surface of the second layer comprises a groove, wherein the channel is formed by the groove and the flat surface of the first layer.

25. The method of claim 24, further comprising bonding a third layer comprising a reservoir to the second and/or first layer, wherein the reservoir is configured to interface with the channel.

26. The method of any one of claim 24 or 25 wherein the bonding is by plasma treatment.
